(19) United States
(12) Patent Application Publication
(10) Pub. No.: US 2007/0179609 A1
(43) Pub. Date: Aug. 2, 2007

(54) THERAPEUTIC AGENT ELUDING IMPLANT WITH PERCUTANEOUS SUPPLY

(75) Inventors: E. Marlowe Goble, Logan, UT (US); Chad W. Lewis, Ogden, UT (US); Daniel F. Justin, Logan, UT (US); M. Mary Sinnott, Logan, UT (US); Daniel E. Gerbec, Logan, UT (US); Karen Mohr, Logan, UT (US); T. Wade Fallin, Hyde Park, UT (US)

Correspondence Address: MEDICINELODGE INC., 180 SOUTH 600 WEST, LOGAN, UT, 84321, US

(73) Assignee: MedicineLodge, Inc., Logan, UT (US)

(21) Appl. No.: 11/627,709

(22) Filed: Jan. 26, 2007

Related U.S. Application Data
(60) Provisional application No. 60/763,069, filed on Jan. 27, 2006.

(51) Int. Cl. A61F 2/28

(52) U.S. Cl. 623/16.11

(57) ABSTRACT

The present invention provides integration between an implant system and therapeutic agent delivery system. The implant may include a prosthesis that restores biomechanical function while decreasing long-term disability and pain by replacing damaged or degenerate tissues, or a reconstructive implant such as a bone plate. The therapeutic agent delivery system may include one or more channels either permanently or reversibly attached to the implant. The channels may receive medication from an external pump via a percutaneous catheter. The channels deliver the medication to one or more medicating surfaces of the implant to treating proximate tissues.

### CROSS-REFERENCE TO RELATED APPLICATIONS

This application claims the benefit of the following:

U.S. Provisional Application No. 60/763,069 filed Jan. 27, 2006, which is entitled THERAPEUTIC AGENT ELUDING IMPLANT WITH PERCUTANEOUS SUPPLY (Applicants' Docket No. MLI-53 PROV).

The foregoing is incorporated herein by reference.

### BACKGROUND OF THE INVENTION

1. The Field of the Invention

The present invention relates generally to systems and methods for supplying therapeutic agents to the area surrounding medical implants through the integration of percutaneous delivery mechanisms with implant structures.

2. The Relevant Technology

Functional restoration of tissue structures is the primary objective of prosthesis applications. Primarily, prostheses successfully retain or replace function, although their application disrupts nearby tissues leading to pain, discomfort, and potentially infections. The current general (e.g., oral route) and local (e.g., regional pain pump) applications of therapeutic agents, such as analgesics and anesthetics, to treat the localized symptoms are known to have unwanted side effects or to ineffectively distribute the therapeutic agent locally around the prosthesis.

Regional pain pumps are currently being used to treat post-surgical discomfort through the manual placement of a percutaneous catheter within the surgical site with or without the use of suture to secure the placement of the catheter tip. Placement of the catheter tip is crucial to the outcome of the treatment. Unfortunately, placement of the catheter tip is highly variable and very cumbersome for the surgeon. Accordingly, the pain medication may be ineffectively delivered, and the process of placing the catheter may add to the patient's discomfort and the length and complexity of the steps carried out by the surgeon.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a perspective view of a therapeutic agent source, a percutaneous therapeutic agent delivery structure, a cutaneous interface, a therapeutic agent interface, and a channel delivery structure.

FIG. 2A is a cross-sectional view of an embodiment of the therapeutic agent interface in FIG. 1, with one channel.

FIG. 2B is a cross-sectional view of an embodiment of the therapeutic agent interface in FIG. 1, with two channels.

FIG. 2C is cross-sectional view of an embodiment of the therapeutic agent interface in FIG. 1, together with a cannula positioned at an entry port.

FIG. 2D is cross-sectional view of an embodiment of the therapeutic agent interface in FIG. 1, together with a cannula introduced into a enclosure.

FIG. 3A is cross-sectional view of an embodiment of the therapeutic agent interface in FIG. 1, together with a balloon-tipped connector positioned at an entry port.

FIG. 3B is a cross-sectional view of an embodiment of the therapeutic agent interface in FIG. 1, together with a balloon-tipped connector introduced into an enclosure, and the balloon partially inflated.

FIG. 3C is a cross-sectional view of an embodiment of the therapeutic agent inter face in FIG. 1, together with a balloon-tipped connector introduced into an enclosure, and the balloon fully inflated.

FIG. 4A is a cross-sectional view of an embodiment of the therapeutic agent interface in FIG. 1, together with a screw-tipped connector.

FIG. 4B is a cross-sectional view of an embodiment of the therapeutic agent interface in FIG. 1, together with a screw-tipped connector introduced into an enclosure.

FIG. 5A is a cross-sectional view of an embodiment of the therapeutic agent interface in FIG. 1, together with a needle-tipped connector positioned at an entry port.

FIG. 5B is a cross-sectional view of an embodiment of the therapeutic agent interface in FIG. 1, together with a needle-tipped connector introduced into an enclosure.

FIG. 6A is a cross-sectional view of a segment of the prosthesis surface, with a circular channel affixed within a groove on the surface, wherein the centroid of the channel is positioned at the surface of the prosthesis.

FIG. 6B is a cross-sectional view of a segment of the prosthesis surface, with a circular channel affixed within a groove on the surface, wherein the centroid of the channel is positioned below the surface of the prosthesis.

FIG. 6C is a cross-sectional view of a segment of the surface of a prosthesis, with a circular channel affixed within a conduit below the surface.

FIG. 6D is a cross-sectional view of a segment of the surface of a prosthesis, with a rectangular channel cut into the surface.

FIG. 6E is a cross-sectional view of a segment of the surface of a prosthesis composed of two parts, with a circular channel below the surface.

FIG. 6F is a cross-sectional view from above of a segment of a prosthesis, with a channel between the upper and lower surfaces of the implant.

FIG. 6G is a cross-sectional view of a segment of the surface of a prosthesis with a semi-circular channel affixed to the surface.

FIG. 6H is a cross-sectional view of a segment of the surface of a prosthesis, with a circular channel below the surface.

FIG. 6I is a cross-sectional view of a segment of the surface of a prosthesis, with a rectangular channel cut into the surface and a layer of material over the conduit.

FIG. 6J is a perspective view of a femoral prosthesis of a knee implant, with subsurface channels according to FIG. 6F.

FIG. 7A is a perspective view of a femoral prosthesis of a knee implant, with which a therapeutic agent delivery structure with channels is affixed via links.

FIG. 7B is a front elevation view of the femoral prosthesis shown in FIG. 7A in position on a patient's knee. A therapeutic agent delivery structure is affixed to the femoral prosthesis, and a therapeutic agent source, a percutaneous therapeutic agent delivery structure, a cutaneous interface, and therapeutic agent interface are connected to the therapeutic agent delivery structure.

FIG. 8A is a cross-sectional view of an embodiment of the link in FIG. 7A, in which a barb-tipped link is positioned outside a chamber on the prosthesis surface to connect a channel to the chamber.

FIG. 8B is a cross-sectional view of an embodiment of the link in FIG. 7A, in which a protrusion-tipped link is positioned outside the boundary between a bone and a prosthesis.

FIG. 8D is a cross-sectional view of an embodiment of the link in the FIG. 7A, in which a protrusion-tipped link is positioned outside an irregularly-edged boundary between a bone and a prosthesis.

FIG. 8E is a cross-sectional view of an embodiment of the link in FIG. 7A, in protrusion-tipped link is positioned outside a chamber on the prosthesis surface.

FIG. 9 is a side elevation view of a knee prosthesis including a therapeutic agent delivery structure.

FIG. 10 is a perspective view of a posterior fusion system including a therapeutic agent delivery structure.

FIG. 11 is a perspective view of an elbow prosthesis including a therapeutic agent delivery structure.

FIG. 12A is a superior perspective view of a breast prosthesis including a therapeutic agent delivery structure.

FIG. 12B is a posterior perspective view of the breast prosthesis of FIG. 12A.

FIG. 13 is a perspective view of a hip prosthesis including a therapeutic agent delivery structure.

FIG. 14A is a perspective view of a bone plate including a therapeutic agent delivery structure.

FIG. 14B is a perspective view of an alternative embodiment of a bone plate including a therapeutic agent delivery structure.

FIG. 15 is a perspective view of a shoulder prosthesis including a therapeutic agent delivery structure.

FIG. 16 is a perspective view of an intervertebral disk implant including a therapeutic agent delivery structure.

FIG. 17 is a perspective view of a calf implant including a therapeutic agent delivery structure.

FIG. 18 is a perspective view of a wrist prosthesis including a therapeutic agent delivery structure.

FIG. 19 is a side elevation view of a cochlear implant including a therapeutic agent delivery structure.

FIG. 20 is a perspective view of an external fixation device fastened in a bone of a patient and including a therapeutic agent delivery structure.

FIG. 21 is a perspective view of an intervertebral body fusion prosthesis including a therapeutic agent delivery structure.

FIG. 22 is a side elevation view of a temporo-mandibular joint prosthesis including therapeutic agent delivery structure.

FIG. 23 is a perspective view of a chin prosthesis including a therapeutic agent delivery structure.

FIG. 24 is a perspective view of an ankle prosthesis including a therapeutic agent delivery structure.

### DETAILED DESCRIPTION OF THE PREFERRED EMBODIMENTS

The present invention provides various configurations of a system in which a therapeutic agent source is connected to a therapeutic agent interface, which in turn is permanently or reversibly connected to one or more channels positioned on or embedded in implant surfaces. Therapeutic agents may be carried from the therapeutic agent source, through the interface, and through the channels to one or more locations on medicating surfaces of the implant. The channels may communicate with a plurality of openings of various diameters through which the therapeutic agents flow and come in contact with the bodily tissues surrounding the implant. With the system installed, a controlled measured flow of a therapeutic agent can pass directly from the channels of the implant to the surrounding bodily tissues, thereby accurately treating only the regional area of concern.

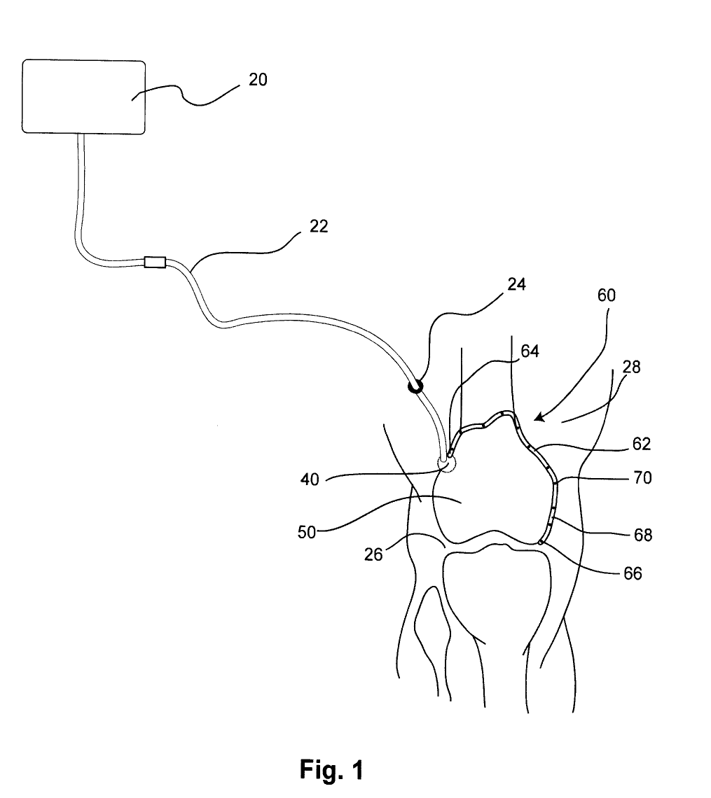

FIG. 1 shows a perspective view of one embodiment of the invention. A therapeutic agent source 20 is connected to a conduit 22, such as a catheter. The conduit 22 percutaneously passes into a patient's body through a cutaneous interface 24, and connects to a therapeutic agent interface 40. A local therapeutic agent delivery structure 60, or structure 60, also connects to the therapeutic agent interface 40, and is adhered to, integrally formed with, or embedded in the body of an implant which has been implanted in a patient's body. In this embodiment, the implant takes the form of a prosthesis 50 configured to replace a bony structure, such as the femoral portion of a knee joint.

The therapeutic agent delivery structure 60 has a channel 62 that originates at a first vend 64 connected to the therapeutic agent interface 40. The channel 62 terminates at a second end 66 along a medicating surface 68 of the prosthesis 50. The medicating surface 68 has, at various intervals, a plurality of openings 70. With the prosthesis 50 in the implanted state, a therapeutic agent can pass from the therapeutic agent source 40, percutaneously through the conduit 22, through the cutaneous interface 24 to the therapeutic agent interface 40. The therapeutic agent is conveyed into the structure 60, along the channel 62, and passes out the openings 70.

In this manner, a therapeutic agent selected by a medical practitioner, such as a chemical agent for alleviating pain, can be dispensed directly to the location of a prosthesis. Such treatment increases the effectiveness of the medication, decreases the potential for unwanted side effects, minimizes the likelihood of infection, and provides a simple medication pathway for any infections that do develop. Additionally, a system according to the present invention can be employed for dispensing other beneficial substances or effects to a prosthesis site. Such substances and effects may include anesthetic agents, analgesic agents, anti-inflammatory agents, anti-rejection agents, growth factors, antibiotics, anti-adhesion factors, saline, glycosaminoglycan varieties, collagen varieties, bio-nutrients, gene-delivery vehicles, stem cells, light, sound, electromagnetic energy, and/or any other therapeutic substance or effect that is desirable to be dispensed to the prosthesis site.

Referring again to FIG. 1, the therapeutic agent source 20 is a mechanism, such as a “pain pump,” that creates a controlled pressure gradient between a therapeutic agent reservoir and a connecting body such as the conduit 22. The conduit 22 may be branching or non-branching, and may be integrally formed with the channels 62, or may be separate from and permanently or reversibly connectable to the channels 62. The medicating surface 68 may be positioned to release the medication into an intra-articular space 26 between the articulating surfaces of the prosthesis 50 and those of an adjacent bone or prosthesis, or to soft tissues 28 proximate the implantation site. In other embodiments, other tissues proximate the implantation site, such as bone tissues, may receive the medication.

Optionally, the conduit 22 and/or the channel 62 may include flow control valves and filters in series along their length. The tubing surface may be treated to increase biocompatibility (e.g., with fluorine or functional groups), block the clotting cascade (e.g., with heparin), provide antimicrobial properties (e.g., with silver), and/or minimize inflammation (e.g., with nitric oxide).

The cutaneous interface 24 shown in FIG. 1 may also be of multiple configurations. In one variation, there is direct contact between the patient dermis and the conduit 22, and the dermis is sutured around the conduit 22 to form the cutaneous interface 24. In other variations, the cutaneous interface 24 is formed by a polymer structure (not shown) that is congruent with patient dermis on its exterior and with one or more conduits 22 passing through the interior surfaces. In one alternative, a specified length of the conduit 22 is encapsulated by the polymer structure to create a congruent interface between the polymer structure and the conduit 22. The dermis is sutured around the exterior surface of the polymer structure.

In another alternative, two parallel conduits 22 of equal length are employed; both lengths may pass through the polymer structure to reach different implant channels, or different portions of a single implant channel. In yet another alternative, the cutaneous interface 24 is formed by a polymer structure that is congruent with the patient dermis on its exterior and with the conduit 22, and with secondary tubing such as aspiration tubing or a power supply cord on the interior surface. Equal lengths of the conduit 22 and the secondary tubing are encapsulated by the polymer structure to create a congruent interface. The dermis is sutured around the exterior surface of the polymer structure.

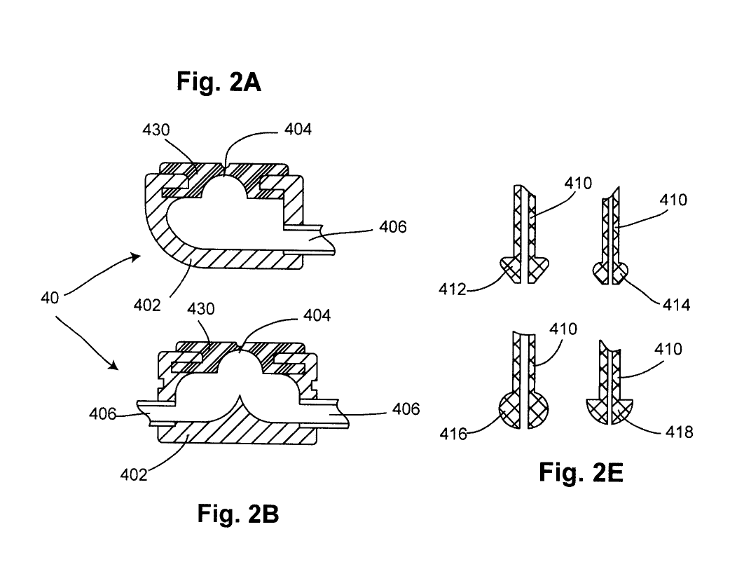

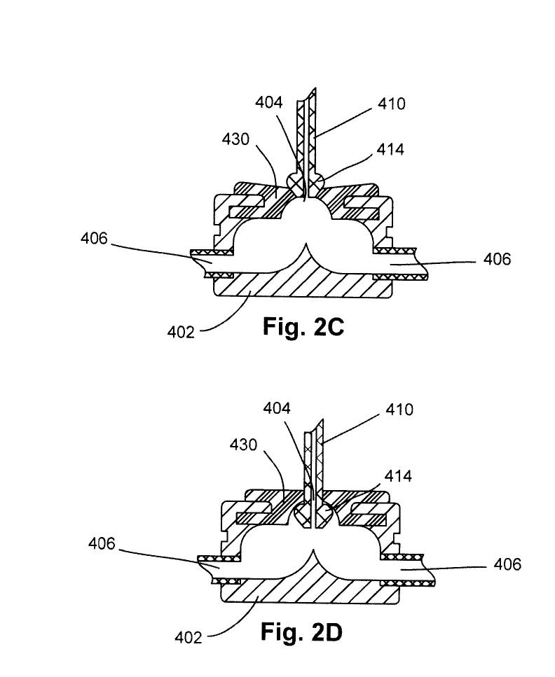

The therapeutic agent interface 40 may be constructed in a variety of designs and from varying materials. FIGS. 2 through 5 illustrate some exemplary embodiments for the therapeutic agent interface 40. Each of the embodiments of FIGS. 2 through 5 may have an enclosure 402, an entry port 404, and one or more openings 406. Any of the embodiments described may have one opening 406, or multiple openings 406, depending on the requirements of the specific application. These illustrations provide only examples and should not be considered to be restrictive of the scope of the invention.

FIGS. 2A through 2E depict various embodiments of the therapeutic agent interface 40, in which the entry port 404 is covered with a reversibly attaching interface 430 and is needleless. An associated cannula 410, which is on the terminus of the conduit 22, can be inserted in the entry port 404 to permit unrestricted therapeutic agent flow between the conduit 22 and the therapeutic agent delivery structure 60. FIG. 2A depicts a therapeutic agent interface 40 with an entry port 404 and one opening 406. Any of the four cannulas 410 shown in FIG. 2E can be inserted in the entry port 404 to permit therapeutic agent flow into the enclosure 402. The therapeutic agent then exits the therapeutic agent interface 40 through the opening 406. FIG. 2B depicts a design identical to FIG. 2A except that two openings 406 are present, allowing for therapeutic agent to flow out of the enclosure 402 in two directions to enter the structure 60, or more precisely, to enter two different channels 62, or two different portions of a single channel 62.

FIG. 2C illustrates a cannula 410 as, it is being inserted into the entry port 404. The heart-shaped tip 414 penetrates the reversibly attaching interface 430, which covers the entry port 404. FIG. 2D illustrates the cannula 410 in place, post-insertion. Therapeutic agent can now flow freely from the therapeutic agent source 20, through the conduit 22, into the therapeutic agent interface 40 via the entry port 404, and out of the therapeutic agent interface 40 through openings 406 into the structure 60. The openings 406 may have different diameters to provide for a greater flow rate of medication to one channel 62, or to one portion of a channel 62.

FIG. 2E illustrates various designs for the tip of the cannula 410. Designs include a triangular tip 412, the heart-shaped tip 414, a speherical tip 416, and a semi-spherical tip 418. These tip designs enhance repeated cannula insertion and removal from the reversibly attaching interface 430 of the entry port 404, while minimizing accidental distraction of the conduit 22 from the therapeutic agent interface 40. The designs pictured in FIG. 2E represent only some of the possible configurations of the cannula 410; other embodiments of the invention may include the alternative tip configurations.

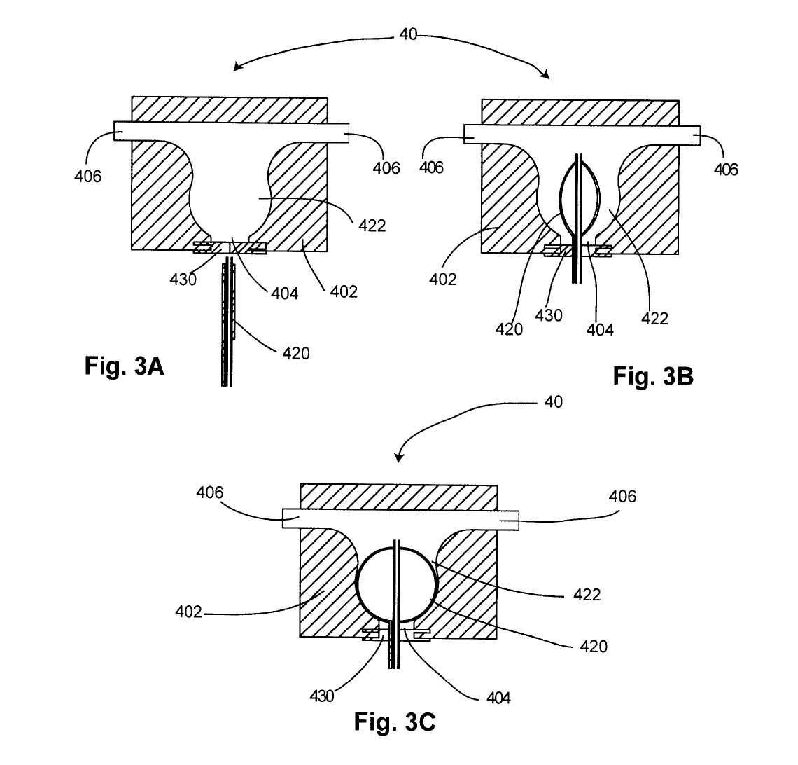

Referring to FIGS. 3A, 3B, and 3C, a balloon-tipped connector 420 is depicted in association with the therapeutic agent interface 40. The enclosure 402 is depicted with a rounded internal cavity 422, one entry port 404 and two openings 406. In FIG. 3A, the balloon-tipped connector 420 is shown prior to insertion into the entry port 404. FIG. 3B depicts the balloon-tipped connector 420 inserted into the entry port 404, with the balloon partially inflated. The balloon-tipped connector 420 may be filled with air or with a liquid, such as saline.

The balloon is fully inflated in the FIG. 3C. The round shape of the internal cavity 422 is congruent to the inflated balloon-tipped connector 420, creating a sealed boundary to the entry port 404. In this state therapeutic agent can flow freely from the conduit 22 (shown in FIG. 1), through the balloon-tipped connector 420 into the enclosure 402, and out of the openings 406. The embodiment of the therapeutic agent interface 430 that permits selective withdrawal of the connector 420 from the internal cavity 422. If a biocompatible liquid is used to fill the balloon of the connector 420, the liquid may simply be released into the internal cavity 422 by rupturing the balloon.

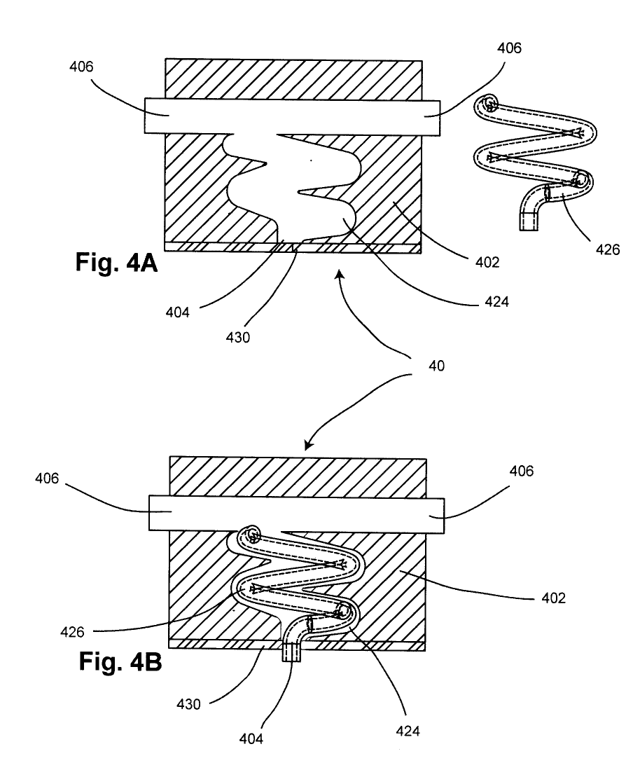

A screw-tipped connector 426 and therapeutic agent interfaces 40 are depicted in FIGS. 4A and 4B. FIG. 4A depicts the empty enclosure 402 with an irregular cavity 424 and a reversibly attaching interface 430 on the entry port 404. The geometry of the screw-tipped connector 426 is configured to mate with the irregular cavity 424 when the tip is inserted and rotated, as shown in FIG. 4B. Once inserted, a sealed boundary to the entry port 404 is created, allowing therapeutic agent to flow freely from the conduit 22 (shown in FIG. 1), through the screw-tipped connector 426 into the enclosure 402, and out of the openings 406. The screw-tipped connector 426 may be removed by rotating it in the opposite direction to permit withdrawal from the irregular cavity 424.

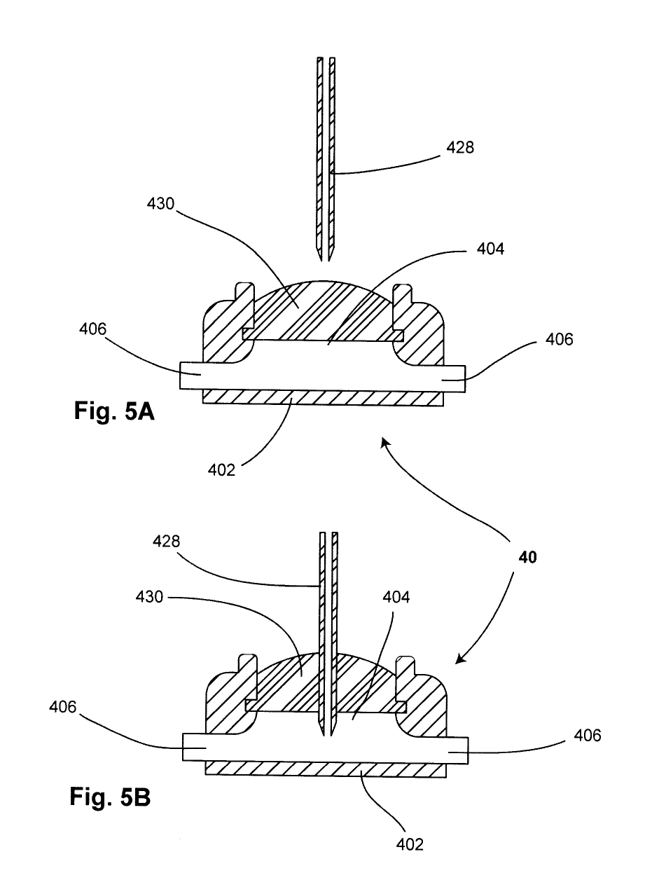

FIGS. 5A and 5B depict a tubular shaped needle cannula 428 and a therapeutic agent interface 40. In FIG. 5A, the bevel-tipped needle cannula 428 is shown before insertion into the entry port 404. FIG. 5B depicts the needle cannula 428 inserted into the reversibly attaching interface 430 on the entry port 404. Thus connected, therapeutic agent can flow freely from the conduit (shown in FIG. 1), through the needle cannula 428 into the enclosure 402, and out of the openings 406. Use of the needle cannula 428 facilitates repeated cannula insertion into and removal from the reversibly attaching interface 430.

The therapeutic agent delivery structure 60 depicted in FIG. 1 can be constructed and configured in a variety of ways. The channels 62 and the openings 70 that comprise the therapeutic agent delivery structure 60 may be composed of the materials which make up the surface of the prosthesis, or may be composed partially or entirely of unlike materials. Furthermore, the structure 60 may be fully or partially embedded within the body of the prosthesis 50 and/or adhered to the surface of the prosthesis 50 via permanent or reversible attachment.

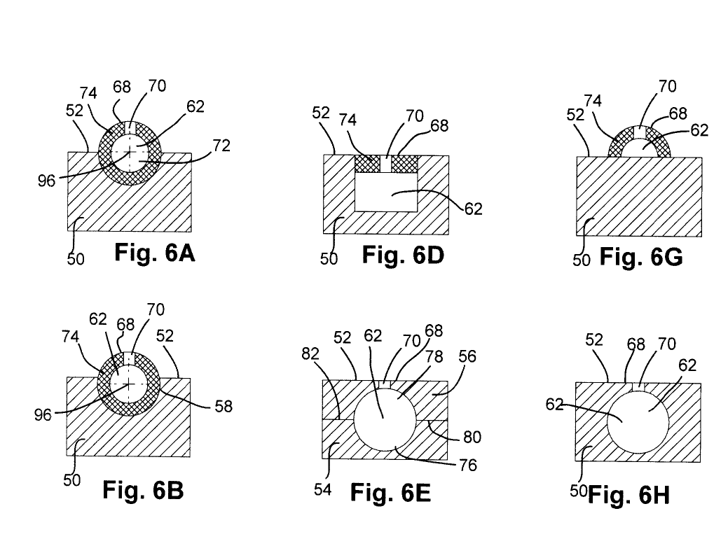

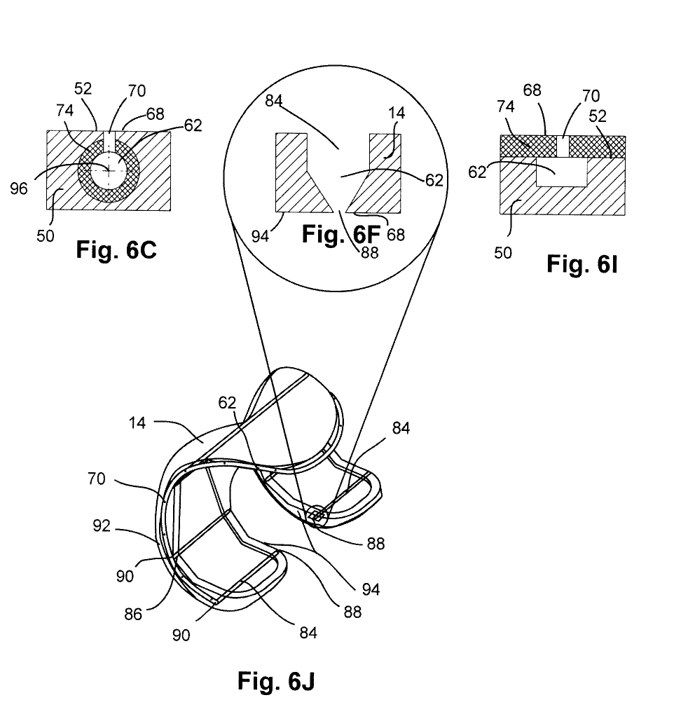

The shape and number of the channel(s) 62 and the shape, number and location of the openings 70 may vary. FIGS. 6A through 6I are cross-sectional views of prosthesis surfaces illustrating possible configurations of the channel 62 and the openings 70 shown in FIG. 1. The variations illustrated in FIGS. 6A through 6I are considered to be illustrative and not restrictive of the scope of the invention.

FIG. 6A illustrates a circular channel 62 which is composed of a material 74 that may be the same as, similar to, or dissimilar to that of the prosthesis 50. The channel 62 of FIG. 6A is partially embedded in the prosthesis 50. The material 74 can either be permanently or reversibly attached using mechanical elements such as snaps, clips, threaded fasteners, and the like, or chemical elements such as biodegradable adhesives. A channel bore 72 is the open area in the channel 62 through which the therapeutic agent is conveyed. In the embodiment pictured in FIG. 6A, the centroid 96 (indicated by dashed lines) of the channel bore 72 is substantially in-plane with a surface 52 of the prosthesis 50. An opening 70 is shown passing through the material 74 from the channel bore 72 to the space outside the channel bore 72.

In the embodiment depicted in FIG. 6B, a circular channel 62 is composed of a material 74 that may be the same as, similar to, or dissimilar to that of the prosthesis 50. The channel 62 of FIG. 6B is partially embedded in a groove 58 on the surface 52 of prosthesis 50. As in the embodiment of FIG. 6A, the material 74 can either be permanently or reversibly attached using mechanical or chemical elements. In this embodiment, the centroid 96 of the channel bore 72 ties below the surface 52 of the prosthesis 50. Because the centroid 96 of the channel bore 72 lies below the surface 52, and the width of the channel 62 is wider than the a edges of the groove 58, the material 74 can be pressed or snapped into the groove 58, and the edges of the groove 58 will aid in retaining the channel 62. An opening 70 is shown passing through the unlike material 74 from the channel bore 72 to the space outside the channel bore 72.

In the embodiment depicted in FIG. 6C, a circular channel 62 is composed of a material 74 that may be the same as, similar to or dissimilar to that of the prosthesis 50. The channel 63 of FIG. 6C is entirely embedded within the prosthesis 50. The sides of the opening 70 are composed partially of the material 74 and partially of the material of which the prosthesis 50 is formed. The centroid 96 of the channel bore 72 lies within the prosthesis 50.

FIG. 6D depicts a channel 62 of a generally rectangular shape, in which three sides of the channel 62 are entirely embedded in the prosthesis 50. A material 74 forms the top side of the channel 62 of FIG. 6D and is flush with the surface 52 of the prosthesis 50. An opening 70 opens out through the material 74. As in previous embodiments, the material 74 may be the same as, similar to, or unlike that of the prosthesis 50, and may be secured through the use of mechanical or chemical elements.

A prosthesis 50 composed of two parts is illustrated in FIG. 6F. A first part 54 has a first partial channel 76 created on a first joining surface 80. A second part 56 has a second partial channel 78 on a second joining surface 82, with an opening 70 passing through the prosthesis surface 52. When the two parts 54, 56 are joined the complete channel 62 is formed between the joining surfaces 80, 82. The channel 62 depicted in this embodiment is circular; however it could be square, rectangular or of any closed shape that can be formed by the joining of the two partial channels 76, 78.

An alternative channel and opening configuration is illustrated in FIGS. 6F and 6J. FIG. 6J displays a perspective view of a femoral prosthesis 14 of a knee replacement system, with an outer edge 92 and an inner edge 94. A channel such as that described in FIG. 6A is affixed to the outer edge 92. The femoral prosthesis 14 has several parallel channels 84 which traverse the prosthesis, below the prosthesis surface by penetration of a drill bit through the prosthesis. Each channel 84 has a first end 86, which is at an outer edge 92 of the femoral prosthesis 14, and a second end 88 which opens at an inner edge 94 of the femoral prosthesis 14.

FIG. 6F displays a cross section of a portion of the channel 84 at the second end 88, as seen from above. The drill bit incompletely penetrates the outer edge 92, so the second end 88 is smaller in diameter than the channel 84. Returning to FIG. 6J, the channel 62 has openings 70, and exit connections 90 at the outer edge 92 where the channel 62 meets the first ends 86 of the channels 84. When therapeutic agent flows into the channels 62, it can flow out the openings 70, and through the exit connections 90, into the channels 84 and out the second ends 88. This configuration of channels 62 and channels 84 allows therapeutic agents to reach the body tissues surrounding the inner edge 94 of the prosthesis as well as the outer edge 92. The relatively small diameter of the second ends 88 may help to control the flow rate of the therapeutic agent from the second ends 88 relative to the flow rate through the openings 70.

FIG. 6G depicts a semi-circular channel 62 lying on the prosthesis surface 52. The channel 62 is composed of a material 74, which may be the same as, similar to, or unlike that of the prosthesis 50. The material 74 may be adhered to the prosthesis surface 52 through the use of mechanical or chemical elements. The openings 70 provide egress for the therapeutic agent through the unlike material 74.

FIG. 6H depicts a circular channel 62 entirely embedded within the prosthesis 50. The channel 62 and opening 70 are formed completely by the surrounding prosthesis 50. Accordingly, no separate structure need be added to form the channel 62.

FIG. 6I depicts a rectangular channel 62 created on the surface 52 of the prosthesis A layer of material 74 seals the top aspect of the channel 62. The material 74 may be the same as, similar to, or unlike that of the prosthesis 50. An opening 70 passes through the unlike material 74 to provide an exit path for the therapeutic agent.

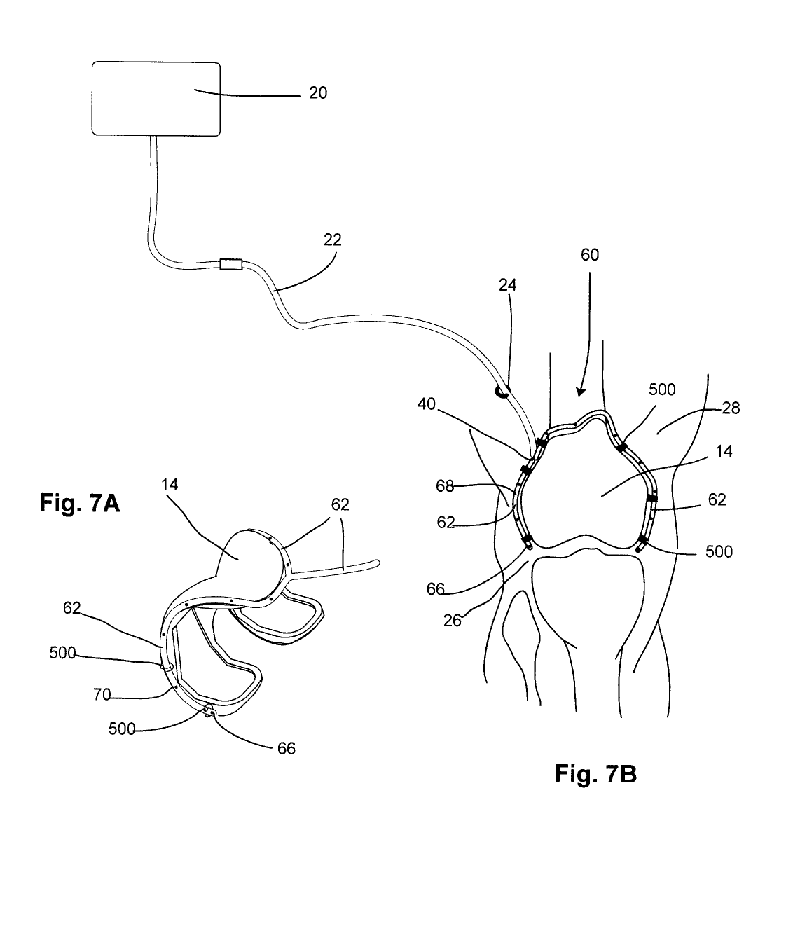

FIG. 7A illustrates channels 62 which lie on or protrude from the surface of a femoral prosthesis 14, such as the channel 62 illustrated in FIG. 6A. A plurality of links 500 holds the channels 62 in place. Each link 500 may permanently or reversibly attach channels 62 and/or conduits 22 to the prosthesis 14. Each link 500 may optionally be biodegradable, and may be formed of a polymer, metal, ceramic, composite, or any combination thereof.

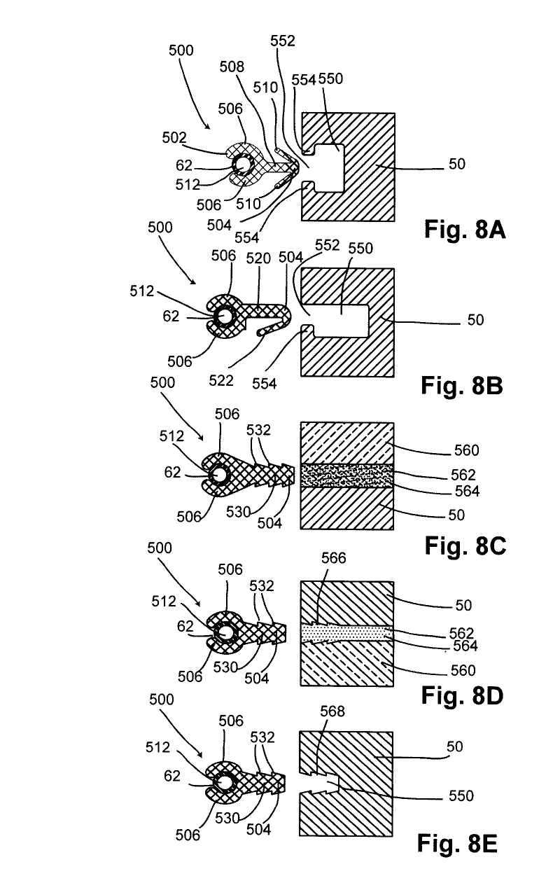

FIG. 7B shows a similar femoral prosthesis 14 in place on a patient's femur, with a therapeutic agent flow structure 60 affixed to the femoral prosthesis. The structure 60 in this example is composed of two channels 62, which extend from the therapeutic agent interface 40 and are held in place by links 500. FIGS. 8A through 8E illustrate a variety of embodiments of the links 500, which may provide permanent or removable attachment of the channels 62 to the prosthesis 14, or to any other medical implant.

In FIG. 8A, a link 500 with a first end 502 and a second end 504 is depicted. The first end 502 terminates in two protruding curved fingers 506, which form a gap 512 with a diameter sized to hold a channel 62. The second end 504 terminates in a barbed tip 508 with two opposing barbs 510. A prosthesis 50 with a chamber 550 is adjacent to the link 500. The chamber 550 opens to the surface of the prosthesis 50 at an opening 552, and two lips 554 extend partially across the opening 550. When the link 500 is inserted through the opening 552 into the chamber 550, the barbs 510 compress to fit through the lips 554. Once inside the chamber 550, the barbs 510 expand back to their original position and prevent the link 500 from coming out of the chamber 550. Either prior to or after attachment of the links 500 to the prosthesis 50, the channel 62 can be pressed into the gap 512 within the curved fingers 506.

A plurality of chambers 550 may be present in the prosthesis 50 to receive a plurality of links 500, alternatively, the chamber 550 may be elongated, so as to form a groove in the prosthesis 50 to receive multiple links 500.

Advantageously, this embodiment permits the surgeon to decide, interoperatively, whether or not to implant the channel 62 with the prosthesis 50. A plurality of channels 62 may be provided to the surgeon, and the surgeon may be able to select from them to optimize characteristics such as the volume of medication delivered, the exact distribution pattern of the medication, and the location at which medication will be delivered. As mentioned previously, the links 500 may be formed of a biodegradable material. Alternatively, the links 500 may be designed to remain in place permanently, or may be made frangible, for example, through the use of a necked-down cross section between the first and second ends 502, 504 to permit the first end 502 to be broken away when removal of the channel 62 is desired. Such variations may also be used with other link embodiments, such as those of FIGS. 8B through 8E.

FIG. 8B illustrates a link 500 with a hooked tip 520 on the second end 504. The hooked tip 520 terminates in a single hook 522. The adjacent prosthesis 50 has a chamber 550 sized to hold the hooked tip 520, and has a single lip 554, which extends partially across an opening 552. Prior to insertion into the opening 552, the link 500 is oriented so the hook 522 is lined up with the lip 554. As the link 500 is inserted through the opening 552, the hook 522 compresses to slide past the lip 554, and then expands back to its original position. Once the link 500 has been inserted, the hook 522 prevents the link 500 from coming out of the chamber 550. Either prior to or after attachment of the links 500 to the prosthesis 50, the channel 62 can be pressed into the gap 512 between the curved fingers 506.

The link 500 illustrated in FIG. 8C is particularly suitable for use when a prosthesis is cemented to a bone. For example, the link 500 of FIG. 8C may be advantageously used with an interbone prosthesis such as a knee, elbow or hip prosthesis. In this application, an “interbone” prosthesis is a prosthesis that operates at the junction of two bones to help facilitate, limit, or otherwise control relative motion between the bones. Thus, interbone prostheses include articulating joints, and also include joints connected by flexible soft tissues without articulating surfaces. An interbone prosthesis may include a prosthesis for each of the adjoining bone structures, or may include only a single prosthesis for one bone of the joint.

Returning to FIG. 8C, the link 500 is shown adjacent to a prosthesis 50, which is being attached to a prepared bone 560. A layer of cement 562 fills a separation 564 between the bone 560 and the prosthesis 50. The link 500 has a second end 504 with a tip 530. A plurality of protrusions 532 encircles the tip 530. These protrusions may be helical protrusions such as common threads or concentric protrusions such as ribs. The protrusions create additional surface area on the outside of the tip 530. When the link 500 is pushed into the separation 564, the cement 562 surrounds the ribbed tip 530 and fills in the spaces between the protrusions 532. When the cement 562 is cured, the link 500 is permanently affixed between the prosthesis 50 and the bone 560. Either prior to or after attachment of the links 500 to the prosthesis 50, the channel 62 can be pressed into the gap 512 between the curved fingers 506.

The link 500 illustrated in FIG. 8D is similar to the link 500 in 8C. However, in this embodiment, the separation 564 between the prosthesis 50 and the prepared bone 560 has helical or concentric edges 566 which are designed to mate with the protrusions 532 on the tip 530. The helical or concentric edges 566 may be tapped so as to mate with helical protrusions, or ribbed to mate with concentric protrusions. When the prosthesis 50 is cemented to the prepared bone 560 with the link 500 in place, the helical or concentric edges 566 will mate with the protrusions 532, making the attachment of the link 500 stronger.

FIG. 8E also displays a link 500 with protrusions 532 on the tip 530. A prosthesis 50 with a chamber 550 is shown next to the link 500. The chamber 500 has helical or concentric sides 568 which are designed to mate with the protrusions 532 on the tip 530. File helical or concentric edges 568 may be tapped so as to mate with helical protrusions, or ribbed to mate with concentric protrusions. When the link 500 is inserted in the chamber 550, the helical or concentric sides 568 will mate with the protrusions 532, preventing the link 500 from coming back out of the chamber 550. Alternatively, the helical or concentric sides 568 may simply resist withdrawal of the protrusions 532 from the chamber 550, thereby requiring the application of a deliberate threshold pullout force before the link 500 will detach from the prosthesis 50.

As another alternative, the channels 62 used may be biodegradable, in addition to or in the alternative to the use of biodegradable links. The channels 62 may simply be formed of a bioabsorbable material, and may be designed to absorb within a time frame longer than that during which the therapeutic agent will be needed. Biodegradable channels 62 may be used with or without biodegradable links.

The embodiment depicted in FIG. 1 illustrates the invention as applied to a knee prosthesis. However, it is appreciated that the invention can be applied to many other implants, including other body part prostheses. For example, the invention may be applied to interbone constrained, semi-constrained or unconstrained joint prostheses such as hip, facet or wrist prostheses. The present invention may alternatively be applied to intrabone implants such as bone plates or rods. It may be applied to percutaneous restorative implants such as an external fixation devices. In addition, it may be applied to other prostheses such as cosmetic implants and artificial organs.

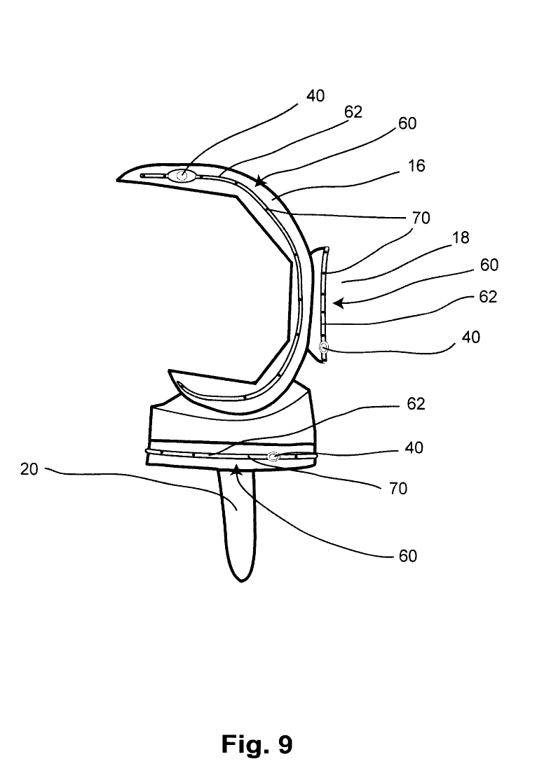

FIGS. 9 through 24 illustrate a variety of alternative applications of the invention. In each illustration, both the therapeutic agent interface 40 and the therapeutic agent delivery structure 60 are depicted as being composed of unlike materials adhered to the outer surface the corresponding prosthesis. However, as discussed above in the descriptions of FIGS. 2 through the therapeutic agent interface 40 may be constructed in a variety of configurations from a variety of biocompatible materials. In addition, as discussed above in the descriptions of FIGS. 6A through 6I, the channels 62 depicted in FIGS. 9 through 24 may be constructed from a variety of materials and may be partially embedded, entirely embedded, or not embedded at all in the surface of the prosthesis. It is appreciated that various features of the above-described examples of therapeutic agent interfaces 40 and channels 62 can be mixed and matched to form a variety of other alternatives, particularly when combined with any of the applications illustrated in FIGS. 9 through 24.

FIG. 9 displays an example of an application of the invention to an interbone prosthesis. A femoral prosthesis 14, a patellar prosthesis 16, and a tibial prosthesis 18 are shown as they would be positioned on a patient's knee. A therapeutic agent interface 40 is positioned adjacent to the femoral prosthesis 14, and a therapeutic agent delivery structure 60 with two channels 62 is positioned on the outer surface of the femoral prosthesis 14. Similarly, a second therapeutic agent interface 40 is positioned adjacent to the patellar prosthesis 16, and a therapeutic agent delivery structure 60 with two channels 62 is positioned around the prosthesis 16. A third therapeutic agent interface 40 is positioned adjacent to the tibial prosthesis 18, and a therapeutic agent delivery structure 60 with two channels 62 is positioned on the tibial prosthesis 18. When a conduit 22 such as depicted in FIG. 1 is connected to each therapeutic agent interface 40, a measured flow of therapeutic agent can be delivered to each therapeutic agent interface 40, into the therapeutic agent delivery structures 60, through the channels 62, and to proximate tissues through the openings 70.

If desired, a single branching conduit (not shown) may be coupled to all three of the therapeutic agent interfaces 40 to deliver therapeutic agents to all three structures 60. Variations in conduit sizing, valves, or the like may be used to control the relative flow rates of therapeutic agents to the structures 60. Alternatively, separate conduits 60 and/or separate therapeutic agent sources 20 may be connected to the three therapeutic agent interfaces 40. Such variations may be used in conjunction with any embodiment of the invention.

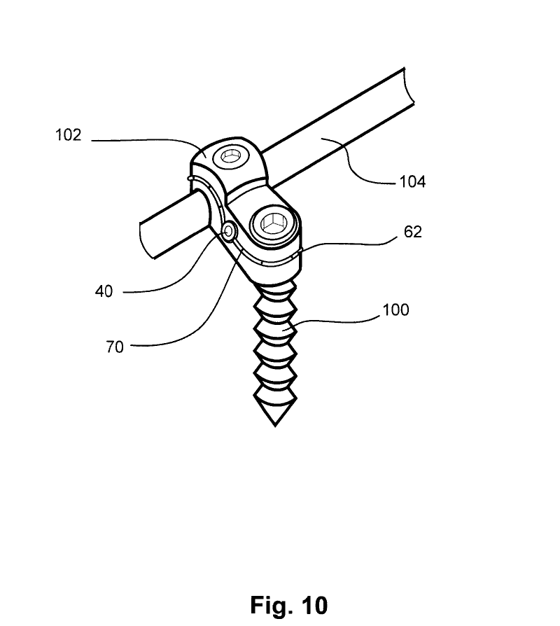

FIG. 10 depicts a perspective view of another interbone prosthesis: a pedicle screw 100 and its associated link body 102 mounted on a rod 104, as in a posterior spinal fixation system. A therapeutic agent delivery structure 60 encircles the outer surface of the link body 102. A therapeutic agent interface 46 lies on the side of the link body 102 and two channels 62 extend from the therapeutic agent interface 40 in opposite directions, terminating on opposite sides of the link body 102. The layout of the therapeutic agent delivery structure 60 depicted is only one possible arrangement; for example the therapeutic agent interface 40 could lie on the top of the link body 102, with multiple channels 62 encircling the top, bottom and sides. Alternatively or additionally, multiple channels 62 may extend between pedicle screws 100 and associated link bodies 102. Structures 60 with channels 62 may additionally or alternatively be coupled to the pedicle screw 100 and/or the rod 104.

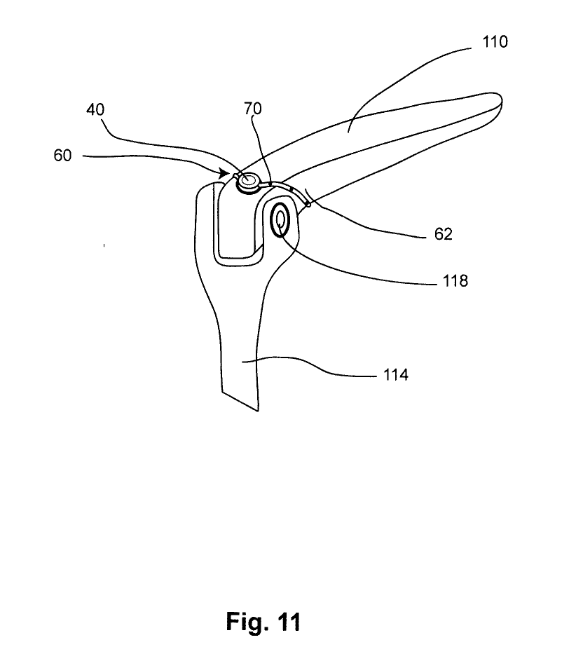

An interbone elbow prosthesis is illustrated in FIG. 11. The prosthesis comprises an ulnar prosthesis 110 and a humeral prosthesis 114. A hinge joint 118 joins the two prosthesis 110, 114. A therapeutic agent delivery structure 60 is affixed to the ulnar prosthesis 110, connecting to a therapeutic agent interface 40 which lies adjacent to the hinge joint 118. Two channels 62 extend in opposite directions from the therapeutic agent interface 40, and encircle the ulnar prosthesis 110. Alternatively or additionally, a therapeutic agent delivery structure 60 could be affixed to the humeral prosthesis 114.

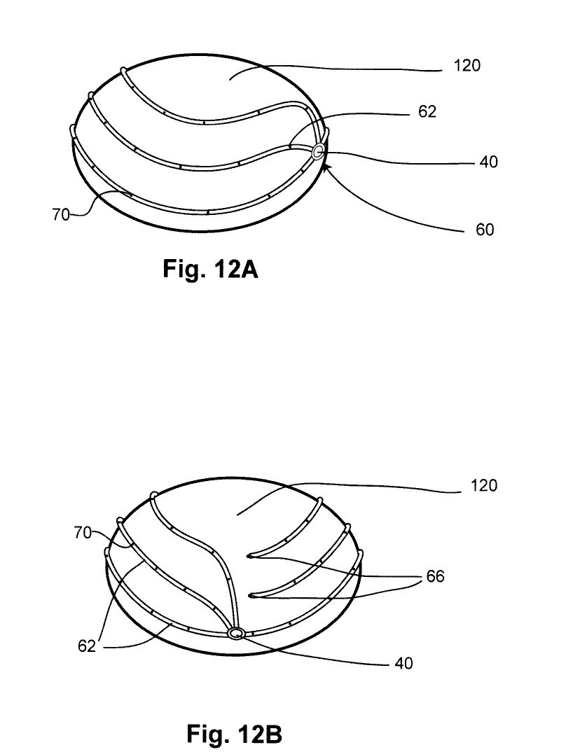

FIGS. 12A and 12B illustrate application of the invention to a restorative or cosmetic prosthesis. FIG. 12A depicts a superior perspective view of a breast prosthesis 120, and FIG. 12B depicts a posterior perspective view of the same prosthesis 120. A therapeutic agent delivery structure 60 is affixed to the prosthesis 120, with a therapeutic agent interface 40 in a posterior location. Multiple channels 62 extend from the therapeutic agent interface 40, encircle the prosthesis 120 and terminate with their second ends 66 also on the posterior side of the prosthesis 120. In this embodiment of the invention, channel configurations which lie below the surface of the prosthesis, such as those pictured in FIGS. 6C, 6D, 6H or 6I, may be preferred, as they would be invisible and not create ridges on the surface of the prosthesis.

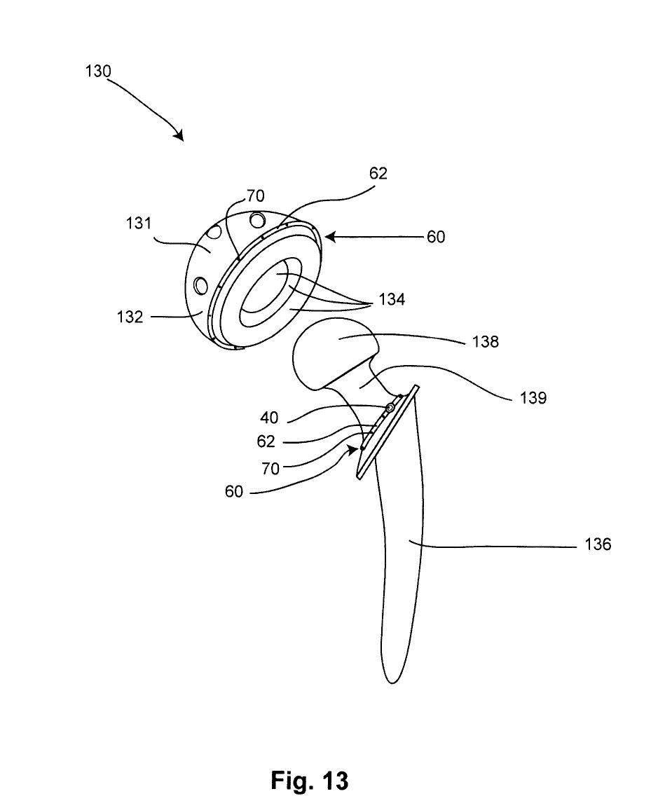

A perspective view of an interbone hip prosthesis 130 is illustrated in FIG. 13. The prosthesis 130 has an acetabular prosthesis 131 with a bearing support 132 and bearing surfaces 134. A femoral prosthesis 135 has a femoral stem 136 and a femoral ball 138 which are joined by a neck 139. One therapeutic agent interface 40 (not visible in FIG. 13) is affixed to the bearing support 132, and a therapeutic agent delivery structure 60 encircles the bearing support prosthesis 132. A second therapeutic agent interface 40 is located on the neck 139, and a second structure 60 encircles the neck 139.

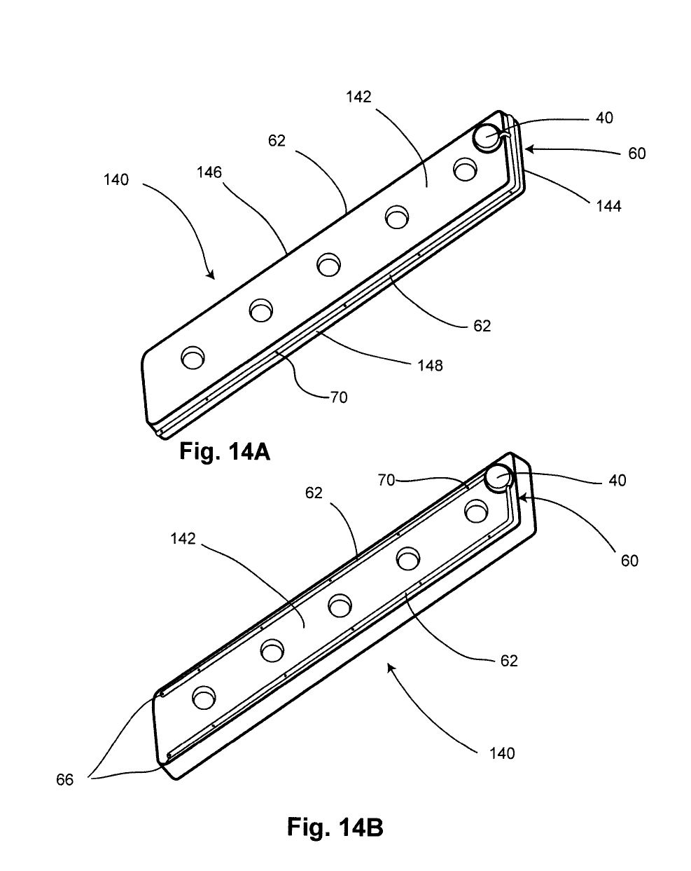

FIG. 14A depicts a perspective view of a bone implant 140 such as a “bone plate,” which is configured to attach to the outside surface of the bone to stabilize a fracture. A therapeutic agent interface 40 lies on an upper surface 142 of the bone prosthesis 140. A therapeutic agent flow structure 60 extends from the therapeutic agent interlace 40 to a first edge 144 and then splits to form two channels 62. The channels 62 run from the first edge 144 around corners on a second edge 146 and a third edge 148. The channels 62 terminate at the ends of the second and third edges 146, 148. Openings 70 release the therapeutic agent to surrounding tissues, such as the fractured bone, surrounding soft tissues, and other tissues that were disturbed during implantation of the bone implant 140.

An alternative arrangement for the therapeutic agent delivery structure 60 is depicted in FIG. 14B. In this arrangement, the therapeutic agent interface 40 lies on the upper surface of the bone prosthesis 140, and two channels 62 extend from the therapeutic agent interface 40 and also lie on the upper surface 142. The two channels 62 extend the length of the bone prosthesis 140 and terminate at their second ends 66. Again, openings 70 release the therapeutic agent to surrounding tissues.

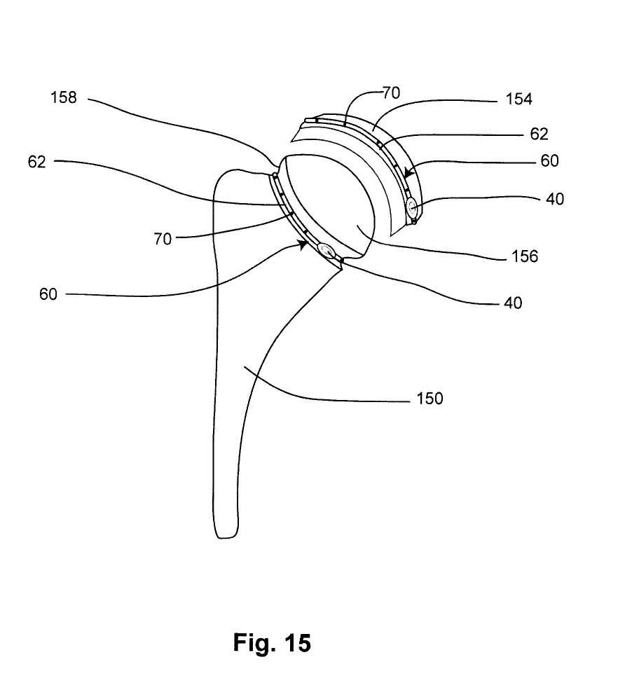

FIG. 15 depicts an interbone shoulder replacement system, which includes a humeral prosthesis 150 with a bearing surface 156 and a separate glenoid prosthesis 154, which is shaped to fit over the bearing surface 156. A therapeutic agent interface 40 and a therapeutic agent delivery structure 60 are positioned on the glenoid prosthesis 154 such that a pair of channels 62 encircles the prosthesis 154. Another therapeutic agent interface 40 and a therapeutic agent delivery structure 60 are positioned on the humeral prosthesis 150, in a trough 158, which lies distal to the bearing surface 156. The therapeutic agent interface 40 lies within the trough 158, as do the channels 62 which extend from the therapeutic agent interface 40 and encircle the humeral prosthesis 150.

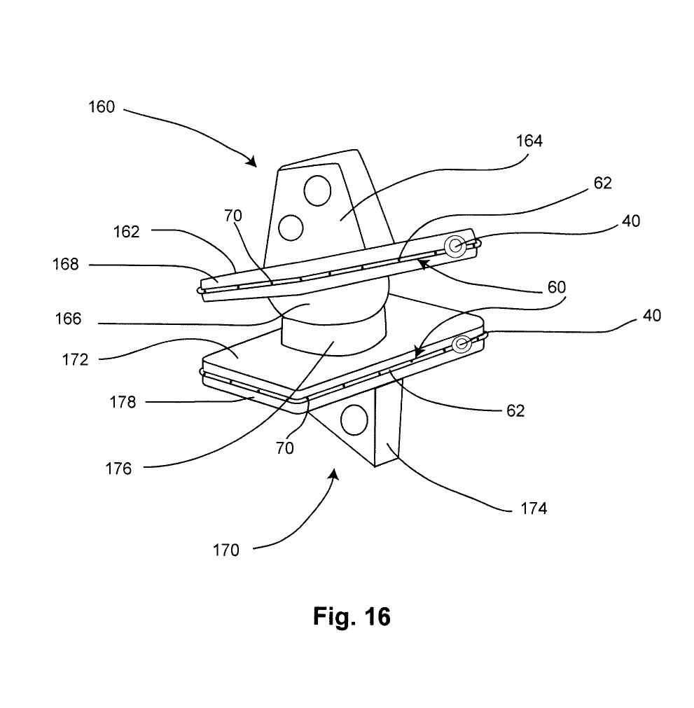

A perspective view of an interbone intervertebral disc replacement system is depicted in FIG. 16. The intervertebral disc replacement system includes a superior prosthesis 160 and an inferior prosthesis 170. The superior prosthesis 160 has a superior endplate 162. On the upper or superior side of the superior endplate 162 is an endplate fixation structure 164, which is designed to be secured to a first vertebral body (not pictured). On the lower or inferior side of the superior endplate 162 is a bearing surface 166. The inferior prosthesis 170 has an inferior endplate 172, with an endplate fixation structure 174, which is designed to be secured to a second vertebral body (not pictured). On its upper or superior side, the inferior endplate 172 has a bearing surface 176, which is designed to lay adjacent to the bearing surface 166 when both the superior 160 and inferior 170 prostheses are implanted.

A therapeutic agent delivery structure 60 is affixed to an outer edge 168 of the superior endplate 162, and a second therapeutic agent delivery structure 60 is affixed to an outer edge 178 of the inferior endplate 172. On each endplate edge 168, 178, a therapeutic agent interface 40 is affixed, and a channel 62 extends out from each lateral side of the therapeutic agent interface 40 to encircle the edge 168, 178.

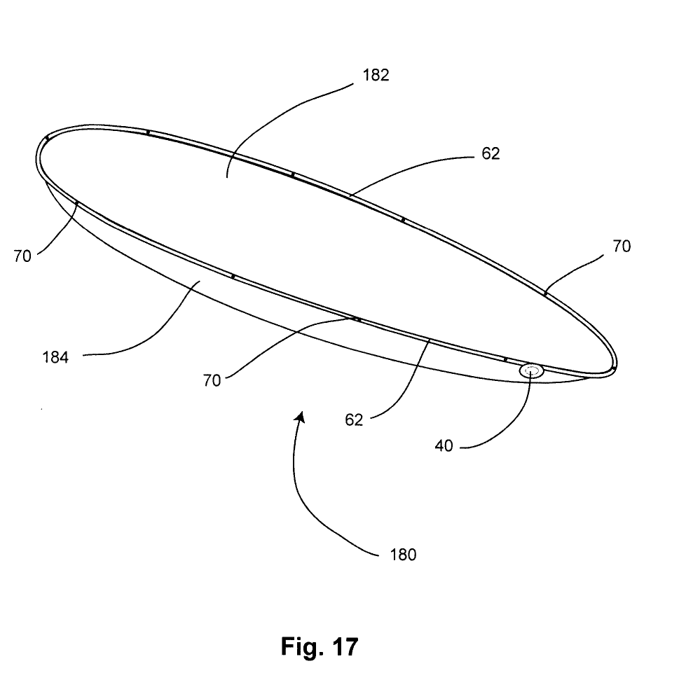

A cosmetic calf implant 180 is depicted in FIG. 17. The implant 180 is generally elliptical in shape with a concave posterior or ventral surface 182, and convex anterior or dorsal surface 184. A therapeutic agent interface 40 is affixed immediately adjacent to the rim 186 on the anterior surface 184, and a channel 62 extends laterally in each direction to encircle the prosthesis on the rim 186.

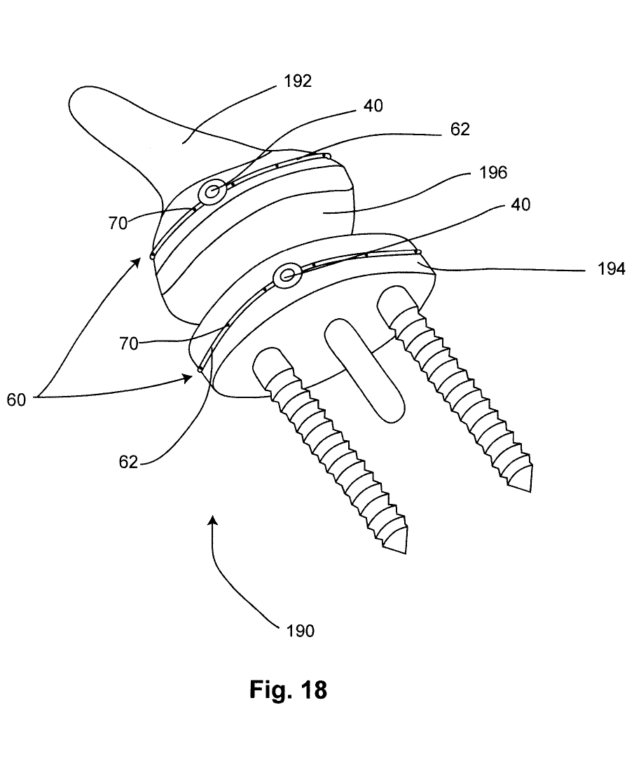

FIG. 18 depicts an interbone wrist prosthesis 190 with two therapeutic agent delivery structures 60 in place. The prosthesis 190 has a distal radius prosthesis 192 and a carpal prosthesis 194, which meet at an artificial articular surface 196. A therapeutic agent interface 40 is affixed to the radius prosthesis 192, and a channel 62 extends laterally in each direction to encircle the radius prosthesis 192, just proximal to the artificial articular surface 196. Similar to the arrangement on the radius prosthesis 192, a therapeutic agent interlace 40 is fixed to the carpal prosthesis 194, and a channel 62 extends laterally in each direction to encircle the carpal prosthesis 194 just distal to the artificial articular surface 196.

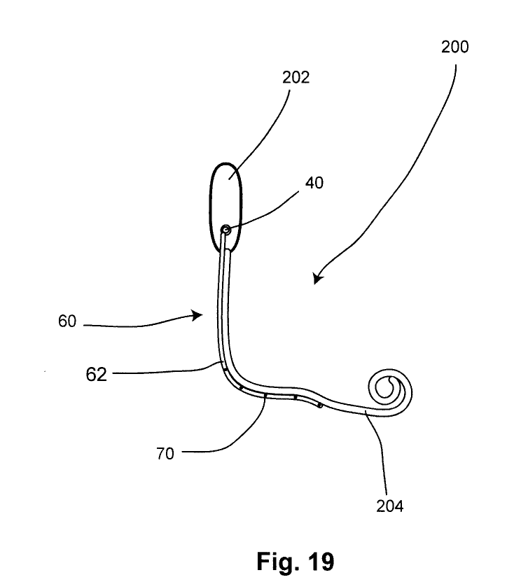

A restorative cochlear implant 200 is illustrated in FIG. 19. The implant 200 has a main body 202, from which extends a long wire-like cochlear electrode 204. A therapeutic agent interface 40 is affixed near one end of the main body 200, and one channel 62 extends along a segment of the length of the cochlear electrode 204.

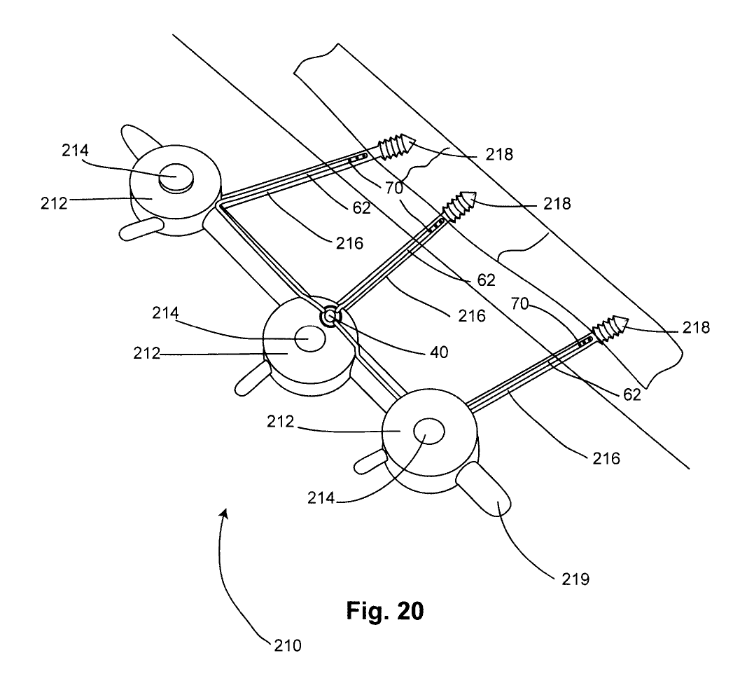

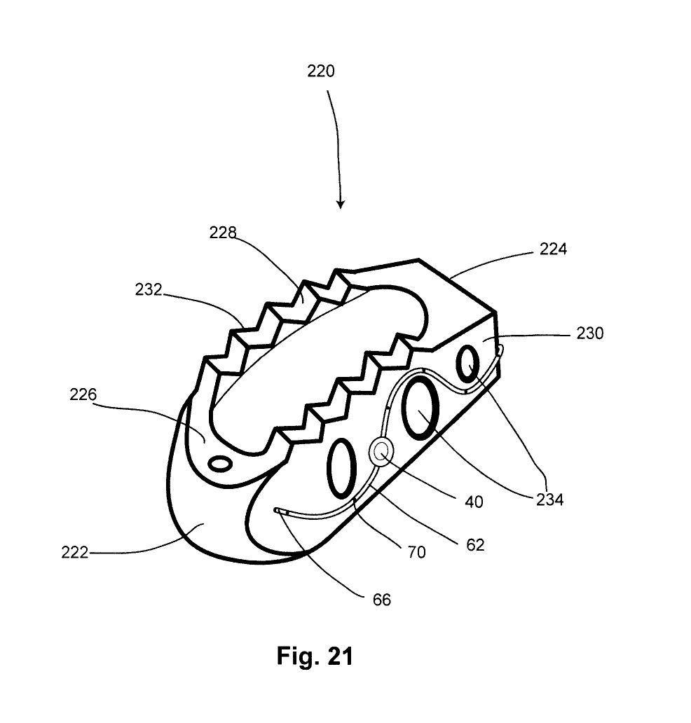

FIG. 20 depicts an intrabone percutaneous external fixation device 210. The device 210 consists of a plurality of circular link bodies 212, which are lined up in a row and joined by a central rod 219. Each link body 212 has an adjustable fastener assembly 214. A bone fixation rod 216 extends laterally from one side of each link body 212, perpendicular to the central rod 219. At the tip of each bone fixation rod 216 is a point 218, which is fixed in a bone segment. In the example in FIG. 20, three link bodies 212 with associated bone fixation rods 216 are depicted. The device 210 may be used to encourage the healing of fractured bone, lengthen a fractured or otherwise damaged bone structure, or perform a variety of other functions.

Attached to the central link body 212 is a therapeutic agent interface 40, from which extends three channels 62. One channel 62 extends directly down the bone fixation rod 216, which is attached to the central link body 212, and the other two channels 62 extend laterally in opposite directions along the central rod 219. When each lateral channel 62 reaches a link body 212, it turns perpendicularly and extends down the associated bone fixation rod 216. Each channel 62 terminates at the base of the point 218. In this embodiment of the invention, the openings 70 are located subcutaneously near the second ends 66 of the channels 62, instead of being evenly distributed along the channels 62. At this location the openings 70 are subcutaneous yet not in the bone.

FIG. 21 illustrates an interbone intervertebral body fusion implant 220. It has an anterior end 222 and a posterior end 224, and a first lateral side 230 and a second lateral side 232. Along a superior side 226 are two rows of toothlike endplate fixation surfaces 228. Located on the lateral sides 230, 232 are a plurality of bone ingrowth spaces 234. A therapeutic agent interface 40 is affixed on the lateral side 230. A channel 62 reaches from each side of the therapeutic agent interface 40 and extends around to both of the lateral sides 230, 232. The channels 62 undulate around the bone ingrowth spaces 234 and terminate with their second ends 66 near the anterior end 222. In this depiction of the invention the therapeutic agent interface 40 is shown the first lateral side 230; however it could be located on the second lateral side 232, the anterior end 222, or the posterior end 224, or even on the interior of the implant 220.

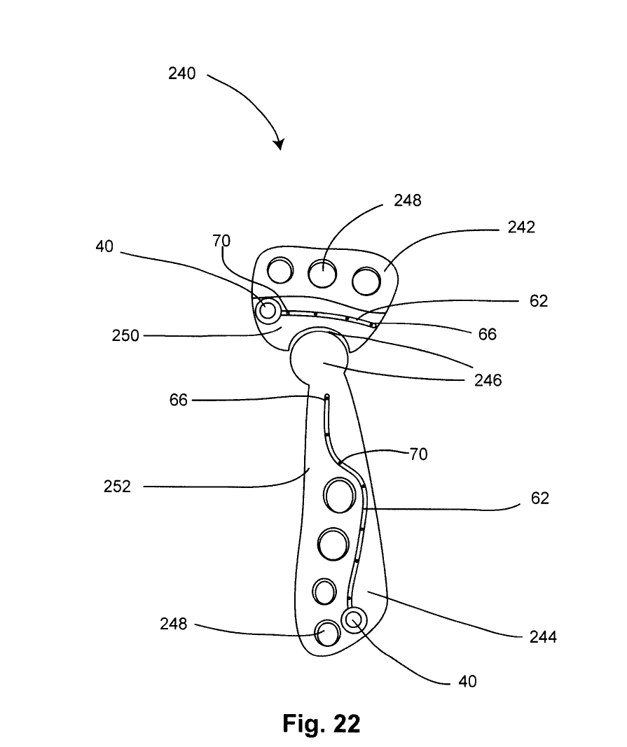

An interbone temporo-mandibular joint prosthesis 240 is depicted in FIG. 22. The temporo-mandibular joint prosthesis 240 comprises an articular fossa prosthesis 242 and a mandibular plate 244 with an artificial articular surface 246. The two prostheses 242, 244 join and articulate at the artificial articular surface 246. Each prosthesis 242, 244 has a plurality of bone screw holes 248. The articular fossa prosthesis 242 has an exterior surface 250, on which a therapeutic agent interface 40 is affixed. A single channel 62 extends across the exterior surface 250 and terminates at a second end 66. The mandibular plate 244 has an exterior surface 252. A therapeutic agent interface 40 is affixed on the distal end of the plate 244, from which a single channel 52 extends in a proximal direction, terminating at a second end 66, near the artificial articular surface 246.

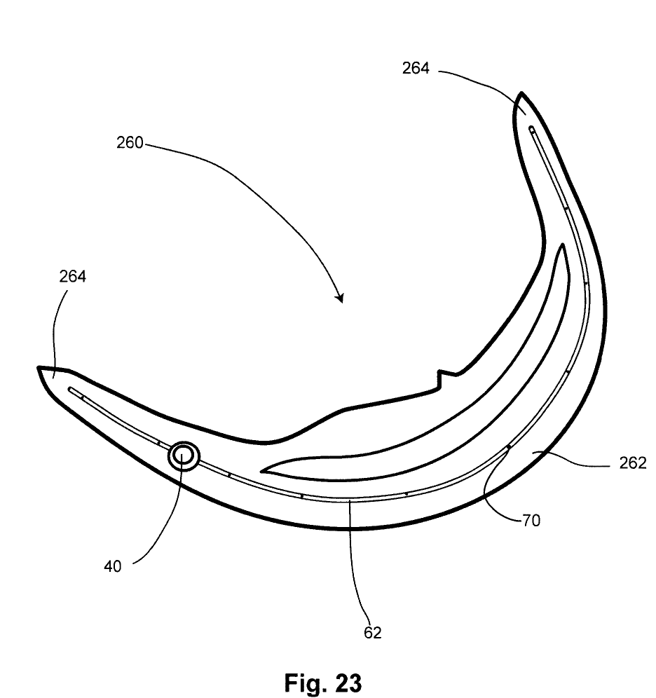

FIG. 23 illustrates a cosmetic or restorative chin implant 260. The implant 260 is generally crescent-shaped, with a central anterior curve 262, which terminates at either end in a prong 264. A therapeutic agent interface 40 is affixed on the implant 260 on one side between the anterior curve 262 and one prong 264. A channel 62 extends from each side of the therapeutic agent interface 40 in each direction. One channel 62 runs from the interlace 40 and terminates near the tip of the closest prong 264, while the other channel runs in the opposite direction from the interface 40, follows the line of the anterior curve 262, and terminates near the tip of the opposing prong 264.

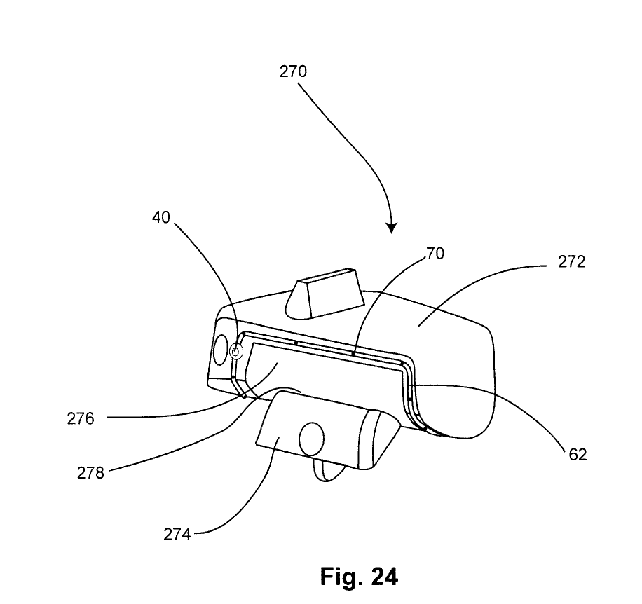

FIG. 24 illustrates an interbone ankle prosthesis 270. The prosthesis 270 comprises a tibial prosthesis 272 and a talar prosthesis 274. The tibial prosthesis 272 is generally U-shaped, with an artificial articular surface 276 on the inside of the U. The talar prosthesis 274 has an elongated wedge shape, designed to fit inside the U formed by the tibial prosthesis 272. An artificial articular surface 278 is located on the outer surface of the talar prosthesis 274, where it contacts the artificial articular surface 276 on the inside of the tibial prosthesis 272. A therapeutic agent interface 40 is affixed to the tibial prosthesis 272, adjacent to, but not on, the artificial articular surface 276. Two channels 62 extend from the therapeutic agent interface 40, and follow the shape of the U such that they outline and lie just outside the artificial articular surface 276.

As indicated previously, FIGS. 9 through 24 provide only a limited set of examples. The principles and structures of the present invention may be used with a wide variety of medical implants, including but not limited to interbone prostheses, non-joint prostheses, reparatory implants, and cosmetic implants, and artificial organs.

The present invention may be embodied in other specific forms without departing from its spirit or essential characteristics. It is appreciated that various features of the above-described examples can be mixed and matched to form a variety of other alternatives. As such, the described embodiments are to be considered in all respects only as illustrative and not restrictive. The scope of the invention is, therefore, indicated by the appended claims rather than by the foregoing description. All changes, which come within the meaning and range of equivalency of the claims, are to be embraced within their scope.

1. A system comprising:
a prosthesis comprising:
a channel shaped to convey a therapeutic agent; and
a medicating surface adjacent to an intra-articular space, the medicating surface having a first opening in communication with the channel to release the therapeutic agent into the intra-articular space.

2. A system as recited in claim 1, further comprising a conduit permanently attached to the prosthesis to percutaneously deliver the therapeutic agent to the channel.

3. A system as recited in claim 2, further comprising a coupling configured to provide a permanent link between the conduit and the channel.

4. A system as recited in claim 1, further comprising a conduit reversibly attached to the prosthesis to percutaneously deliver the therapeutic agent to the channel.

5. A system as recited in claim 4, further comprising a coupling configured to provide a removable link between the conduit and the channel.

6. A system as recited in claim 4, further comprising a coupling configured to provide a biodegradable link between the conduit and the channel.

7. A system as recited in claim 1, wherein the channel comprises a centroid positioned substantially collinear with an exterior surface of the prosthesis.

8. A system as recited in claim 1, wherein the channel comprises a centroid positioned outside an exterior surface of the prosthesis.

9. A system as recited in claim 1, wherein the medicating surface further comprises a second opening having a size different from a size of the first opening.

10. A system as recited in claim 1, further comprising a conduit configured to be captured by the channel to percutaneously deliver the therapeutic agent to the first opening.

11. A system as recited in claim 1, wherein the prosthesis is selected from the group consisting of a knee prosthesis, a hip prosthesis, an ankle prosthesis, a shoulder prosthesis, an artificial spinal disc, an intervertebral fusion prosthesis, a facet prosthesis, an elbow prosthesis, and a temporomandibular joint prosthesis.

12. A system as recited in claim 11, wherein the prosthesis comprises a knee prosthesis configured to replace at least one articular surface of a knee.

13. A method for treating a joint, the method comprising:
preparing a surface of a bone of the joint;
attaching a prosthesis to the prepared bone surface, the prosthesis comprising a channel shaped to convey a therapeutic agent, and a medicating surface having a first opening in communication with the channel;
wherein attaching the prosthesis to the prepared bone surface comprises positioning the first opening to release a therapeutic agent into the intra-articular space of the joint.

14. The method of claim 13, further comprising urging the therapeutic agent to flow percutaneously through a conduit permanently attached to the prosthesis, into the channel from the conduit, and into the intra-articular space via the first opening.

15. The method of claim 13, further comprising:
reversibly attaching a conduit to the prosthesis; and
urging the therapeutic agent to flow percutaneously through the conduit, into the channel from the conduit, and into the intra-articular space via the first opening.

16. The method of claim 13, further comprising urging the therapeutic agent to flow percutaneously through a conduit captured by the channel, into the channel, and into the intra-articular space via the first opening.

17. The method of claim 13, wherein the joint comprises a knee, wherein attaching the prosthesis to the prepared bone surface comprises replacing at least one articular surface of the knee.

18. A system comprising:
a prosthesis configured to articulate with an adjacent bone or implant, the prosthesis comprising:
a channel shaped to convey a therapeutic agent; and
a medicating surface in contact with soft tissue, the medicating surface having a first opening in communication with the channel to release the therapeutic agent to the soft tissue.

19. A system as recited in claim 18, further comprising a conduit permanently attached to the prosthesis to percutaneously deliver the therapeutic agent to the channel.

20. A system as recited in claim 19, further comprising a coupling configured to provide a permanent link between the conduit and the channel.

21. A system as recited in claim 18, further comprising a conduit reversibly attached to the prosthesis to percutaneously deliver the therapeutic agent to the channel.

22. A system as recited in claim 21, further comprising a coupling configured to provide a removable link between the conduit and the channel.

23. A system as recited in claim 21, further comprising a coupling configured to provide a biodegradable link between the conduit and the channel.

24. A system as recited in claim 18, wherein the channel comprises a centroid positioned substantially collinear with an exterior surface of the prosthesis.

25. A system as recited in claim 18, wherein the channel comprises a centroid positioned outside an exterior surface of the prosthesis.

26. A system as recited in claim 18, wherein the medicating surface further comprises a second opening having a size different from a size of the first opening.

27. A system as recited in claim 18, further comprising a conduit configured to be captured by the channel to percutaneously deliver the therapeutic agent to the first opening.

28. A system as recited in claim 18, wherein the prosthesis is selected from the group consisting of a knee prosthesis, a hip prosthesis, an ankle prosthesis, a shoulder prosthesis, an artificial spinal disc, an intervertebral fusion prosthesis, a facet prosthesis, an elbow prosthesis, and a temporomandibular joint prosthesis.

29. A system as recited in claim 28, wherein the prosthesis comprises a knee prosthesis configured to replace at least one articular surface of a knee.

30. A method for treating a joint, the method comprising:
preparing a surface of a bone of the joint;
attaching a prosthesis to the prepared bone surface, the prosthesis comprising a channel shaped to convey a therapeutic agent, and a medicating surface having a first opening in communication with the channel;
wherein the prosthesis is configured to replace at least one articular surface of the joint;
wherein attaching the prosthesis to the prepared bone surface comprises positioning the first opening to release a therapeutic agent to the soft tissue.

31. The method of claim 30, further comprising urging the therapeutic agent to flow percutaneously through a conduit permanently attached to the prosthesis, into the channel from the conduit, and into the intra-articular space via the first opening.

32. The method of claim 30, further comprising:
reversibly attaching a conduit to the prosthesis: and
urging the therapeutic agent to flow percutaneously through the conduit, into the channel from the conduit, and into the intra-articular space via the first opening.

33. The method of claim 30, further comprising urging the therapeutic agent to flow percutaneously through a conduit captured by the channel, into the channel, and into the intra-articular space via the first opening.

34. The method of claim 30, wherein the joint comprises a knee, wherein attaching the prosthesis to the prepared bone surface comprises replacing at least one articular surface of the knee.

35. A system comprising:
an implant configured to attach to an outer surface of a bone to span a fracture of the bone to facilitate healing of the fracture, the implant comprising:
a channel shaped to convey a therapeutic agent; and
a medicating surface having an opening in communication with the channel to release the therapeutic agent.

36. A system as recited in claim 35, further comprising a conduit permanently attached to the implant to percutaneously deliver the therapeutic agent to the channel.

37. A system as recited in claim 35, further comprising a conduit reversibly attached to the implant to percutaneously deliver the therapeutic agent to the channel.

38. A system as recited in claim 35, wherein the channel comprises a centroid positioned substantially collinear with an exterior surface of the implant.

39. A system as recited in claim 35, wherein the channel comprises a centroid positioned outside an exterior surface of the implant.

40. A system as recited in claim 35, wherein the medicating surface further comprises a second opening having a size different from a size of the first opening.

41. A system as recited in claim 35, further comprising a conduit configured to be captured by the channel to percutaneously deliver the therapeutic agent to the first opening.

42. A method for treating a bone fracture, the method comprising:
positioning an implant on a surface of the bone such that the implant spans the fracture, the implant comprising a channel shaped to convey a therapeutic agent, and a medicating surface having a first opening in communication with the channel; and
attaching the implant to the prepared bone surface.

43. The method of claim 42, further comprising urging the therapeutic agent to flow percutaneously through a conduit permanently attached to the implant, into the channel from the conduit, and to proximate tissue via the first opening.

44. The method of claim 42, further comprising:
reversibly attaching a conduit to the implant; and
urging the therapeutic agent to flow percutaneously though the conduit, into the channel from the conduit, and to proximate tissue via the first opening.

45. The method of claim 42, further comprising urging the therapeutic agent to flow percutaneously through a conduit captured by the channel, into the channel, and to proximate tissue via the first opening.
